(19) United States
(12) Patent Application Publication
(10) Pub. No.: US 2019/0142948 A1
(43) Pub. Date: May 16, 2019

(54) AQUEOUS BASED CAPSAICINOID FORMULATIONS AND METHODS OF MANUFACTURE AND USE

(71) Applicant: VIZURI HEALTH SCIENCES LLC, Fairfax, VA (US)

(72) Inventors: Charles A. BIRBARA, Worcester, MA (US); Philip J. BIRBARA, West Hartford, CT (US); Daniel BUCKS, Millbrae, CA (US); Mary COUGHLIN, Worcester, MA (US)

(21) Appl. No.: 16/244,896

(22) Filed: Jan. 10, 2019

Related U.S. Application Data
(63) Continuation of application No. 14/253,660, filed on Apr. 15, 2014, now abandoned.
Application No. 14/253,660 is a continuation-in-part of application No. 14/078,253, filed on Nov. 12, 2013, now abandoned.

(51) Int. Cl. A61K 47/36; A61K 9/00; A61K 31/728; A61K 31/167; A61K 31/085; A61K 31/165; A61K 31/045; A61K 31/05; A61K 45/06

(52) U.S. Cl. CPC A61K 47/36; A61K 9/0024; A61K 31/728; A61K 31/167; A61K 45/06; A61K 31/165; A61K 9/0019; A61K 31/045; A61K 31/05; A61K 31/085

(57) ABSTRACT

Capsaicinoid formulations and methods of treatment are disclosed herein which can be utilized to treat/attenuate pain in mammals. Typically, administration is via injection at a discrete site to provide pain relief for an extended period of time. The formulations are administered in a pharmaceutically acceptable vehicle. The formulations include an analgesic agent in an amount sufficient to attenuate the burning and hyperalgesic effects of capsaicinoid administration. The invention also includes a method of treating pain by administering a corticosteroid followed by administration of a capsaicinoid.

### CROSS-REFERENCE TO RELATED APPLICATION

This application is a continuation of U.S. application Ser. No. 14/253,660, filed Apr. 15, 2014, which is a continuation-in-part of U.S. application Ser. No. 14/078,253, filed Nov. 12, 2013, the contents of each of which are hereby incorporated by reference.

### FIELD OF THE INVENTION

The present invention is directed to compositions for the administration of a capsaicinoid into localized areas. The compositions are useful for the treatment of multiple disorders involving pain (acute and chronic, moderate to severe), and they provide extended pain relief. The invention also relates to methods including pretreatments which limit burning and stinging pain associated with capsaicinoid injection.

### BACKGROUND OF THE INVENTION

Capsaicin, a pungent substance in the fruit of capsicum plants, works to relieve pain by causing a localized degradation of the C neuron endings; capsaicinoids being the only analgesics known to relieve pain by this mechanism. The activity of capsaicin results from its binding to, and activating, an ion channel called vanilloid receptor 1 (VR1). Under normal circumstances, when the VR1 ion channel is activated it opens for a short time, causing the C neurons to transmit a pain signal toward the brain. When capsaicin binds to, and activates VR1, it causes a series of events within the cell that degrade the C neural endings, or terminals of the C neuron, thereby preventing the neuron from transmitting pain signals.

Although capsaicin's analgesic effect was thought to be due to depletion of the substance P, recent evidence suggests a process of “defunctionalization” of nociceptor fibers as being responsible for the analgesic effect of capsaicin. (Anand P, Bley K. Topical capsaicin for pain management: therapeutic potential and mechanisms of action of the new high-concentration capsaicin 8% patch. Br J Anaesth. 2011; 107(4):490-502.)

Capsaicin topical ointments and creams relieve minor aches and pains of muscles and joints. Capsaicin is currently marketed as over-the-counter topically applied, non-sterile creams and patches containing capsaicin at low doses. Concentrations of capsaicin are typically between 0.025 wt. % and 0.075 wt. %. These over-the-counter topical preparations are used topically by consumers to relieve pain with variable and often inadequate results when used to treat conditions such as osteoarthritis, shingles (herpes zoster), psoriasis and diabetic neuropathy. Capsaicin is also available in large adhesive bandages that can be applied to the back.

Topical preparations of capsaicin are used in a variety of skin disorders that involve pain and itching, such as post herpetic neuralgia, diabetic neuropathy, prorates, psoriasis, cluster headache, post mastectomy pain syndrome, rhinopathy, oral mucositis, cutaneous allergy, detrusor hyperreflexia, loin pain/hematuria syndrome, neck pain, amputation stump pain, reflex sympathetic dystrophy, skin tumor, arthritis including rheumatoid arthritis and osteoarthritis, post-surgical pain, oral pain, and pain caused by injury, amongst others. (Martin Hautkappe et al., Review of the Effectiveness of Capsaicin for Painful Cutaneous Disorders and Neural Dysfunction, Clin. J. Pain, 14:97-106, 1998).

Hyaluronic acid (HA) or hyaluronan is a glycosaminoglycan constituent of synovial fluid. HA is responsible for the viscoelastic quality of synovial fluid that acts as both a lubricant and shock absorber. Injection of HA preparations into the knee and hip is commonly used to treat osteoarthritis, but there is debate over the efficacy of the treatment and benefit-to-risk ratio. A Cochrane review of 40 placebo-controlled trials with five different hyaluronan products found statistically significant improvements in pain on weight bearing when results were pooled, but improvements were variable.

Injections of hyaluronic acid (such as Supartz, Hyalgan, Synvisc, Artzal, and Nuflexxa) into the joint—a procedure called viscosupplementation—can provide pain relief for knee osteoarthritis. No major safety issues were detected, but in placebo-controlled trials minor adverse events such as transient pain at the injection site occurred slightly more frequently in patients treated with intra-articular hyaluronan than in those treated with intra-articular corticosteroids. (Zhang et al,. OARSI recommendations for the management of hip and knee osteoarthritis, part II: OARSI evidence-based, expert consensus guidelines. Osteoarthritis Cartilage 2008; 16:137-162.)

Further, HA has been shown to induce analgesia in a bradykinin-induced model of joint pain in rats. This analgesic action was also molecular weight (MW)-dependent, as significant effects were observed at lower concentrations with a higher-MW formulation than with lower-MW HAs. (Gotoh Set al: Effects of the molecular weight of hyaluronic acid and its action mechanisms on experimental joint pain in rats. Ann Rheum Dis 1993, 52:817-822).

HA may have direct or indirect effects on substance P, which can be involved in pain. Since substance P interacts with excitatory amino acids, prostaglandins, and NO, the effects of HA on these factors can indirectly affect the pharmacology of substance P. Additionally, HA has been shown to inhibit an increased vascular permeability induced by substance P. (Moore et al: Hyaluronan as a drug delivery system for diclofenac: a hypothesis for mode of action. Int J Tissue React 1995, 17:153-156.)

Anti-inflammatories decrease the inflammation and swelling at the tissue site. They do this by blocking certain physiologically active substances. For example, these substances, prostaglandin, substance P and histamine, can cause small arteries to dilate with subsequent edema or fluid formation. Soft tissue, muscle, and nerve cells become irritable inflamed and hyper excitable. Anti-inflammatory medications can inhibit this inflammatory process. The treatment of chronic pain with anti-inflammatories however, is limited.

The mainstays for pain relief after total hip arthroplasty and total knee arthroplasty have been the opioids. Although these medications are excellent analgesics, opioids have problems that limit their effectiveness. Alternative analgesics have been considered too mild for the pain caused by these procedures.

U.S. Pat. No. 5,962,532 discloses methods and compositions for treating pain at a specific site with an effective concentration of capsaicin or analogues thereof. The methods involve providing anesthesia to the site where the capsaicin or analogues thereof is to be administered, and then administering an effective concentration of capsaicin to the joint.

U.S. Pat. No. 8,158,682 relates to methods for treating or attenuating pain in a patient. Specifically, the invention provides a method for attenuating pain in proximity to the site of an open wound or surgical incision comprising instilling a pharmaceutical composition comprising a capsaicinoid into the wound or incision, allowing the pharmaceutical composition to dwell for a predetermined period of time, and aspirating the wound or incision to remove the pharmaceutical composition. The invention also provides a method for attenuating pain in proximity to a joint comprising intra-articularly injecting a pharmaceutical composition comprising a capsaicinoid into the joint, allowing the pharmaceutical composition to dwell for predetermined period of time, and aspirating the joint to remove the pharmaceutical composition. In certain embodiments of the invention, the capsaicinoid is capsaicin.

U.S. Pat. No. 8,420,600 discloses compositions and methods for relieving pain at a site in a human or animal by administering at a discrete site in a human or animal a dose of capsaicin in an amount effective to denervate the discrete site without eliciting an effect outside the discrete location.

US 2004/0161481 discloses a method for relieving pain at a site in a human or animal in need thereof, comprising administering by injection or infiltration, a dose of a capsaicinoid and coadministering an effective amount of a NSAID to decrease an undesired effect of the capsaicinoid.

US 2004/0156931discloses a method for relieving pain at a site in a human or animal in need thereof, comprising administering by injection or infiltration, a dose of a capsaicinoid and co administering a vasodilator.

US 2004/0186182 discloses a method for relieving pain at a site in a human or animal in need thereof, comprising administering by injection or infiltration, a dose of a capsaicinoid and co administering a non-anesthetic sodium channel blocker.

US 2005/0019436 discloses compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US 2005/0020690 discloses compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

US 2005/0058734 discloses a method for relieving pain at a site in a human or animal in need thereof, comprising administering by injection or infiltration, a dose of a capsaicinoid and co administering a vasoconstrictor.

US 2006/0269628 discloses compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

U.S. Patent Application 2006/0100272 discloses compositions and methods for the treatment of pain, and neuropathic pain in particular.

US 2006/0148903 provides capsaicinoid gel formulations and methods for relieving pre- and post-surgical pain at a site in a human or animal by administering at a surgical site in a human or animal in need thereof a dose of capsaicinoid gel in an amount effective to attenuate post-surgical pain at the surgical site, the dose of capsaicin ranging from 100 μg to 10,000 μg.

US 2007/0293703 provides methods for synthesizing the trans isomer of capsaicin and/or capsaicin-like compounds by utilizing a process wherein the trans geometry is set from the beginning of the synthesis reaction and carried through the entire synthesis process.

US 2008/0153780 relates to the use of a vanilloid receptor agonist together with a glycosaminoglycan or proteoglycan for producing an agent for treating pain.

US 2007/0036876 provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US 2008/0260791 provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 5000 μg.

US 2008/0262091 A1 provides compositions and methods for attenuating or relieving pain at a site in a human or animal in need thereof by infiltrating at a surgical site or open wound in a human or animal a dose of capsaicinoid in an amount effective to denervate the surgical site or open wound substantially without eliciting an effect outside the surgical site or open wound.

US 2009/0062359 relates to a method for relieving pain at a site in a human or animal in need thereof, comprising administering by injection or infiltration, a dose of a capsaicinoid and co administering a non-anesthetic sodium channel blocker.

US 2009/0054527 discloses a method for relieving pain at a site in a human or animal in need thereof, comprising administering by injection or infiltration, a dose of a capsaicinoid and co administering a vasodilator.

US 2009/0111792 discloses a method for relieving pain at a site in a human or animal in need thereof, comprising administering by injection or infiltration, a dose of a capsaicinoid and co administering a tricyclic antidepressant.

US 2009/0117167 Al discloses a method for relieving pain at a site in a human or animal in need thereof, comprising administering by injection or infiltration, a dose of a capsaicinoid and co administering a vasoconstrictor.

US 2009/0118242 discloses a method for relieving pain at a site in a human or animal in need thereof, comprising administering by injection or infiltration, a dose of a capsaicinoid and co administering an effective amount of a NSAID to decrease an undesired effect of the capsaicinoid.

US 2011/0311592 teaches methods of increasing solubility of poorly soluble compounds and methods of making and using formulations of such compounds.

ALGRX-4975 was in clinical developed to treat the pain associated with osteoarthritis, tendonitis and postsurgical conditions, as well as for neuropathic pain occurring secondary to nerve injury and other chronic pain conditions. By targeting only the C neuron pain fibers, ALGRX-4975 was able to produce significant long-term analgesia. In clinical studies, ALGRX-4975 has provided long-term relief of pain from a single treatment as summarized in TABLE 1,

TABLE I
A SUMMARY OF ALGRX-4975 CLINICAL TRAILS

| ALGRX-4975 | | |
|---|---|---|
| Volume | Concentration | |
| (ml) | (mg/ml) | COMMENTS |
| 4 | 0.25 | ⁽¹⁾Significant pain reduction of mean Visual |
| | | Analogue Scale (VAS) at 8 hr and 24 hr post |
| | | unilateral bunionectomy after a single intra- |
| | | operative instillation. |
| 0.5 | 0.2 | ⁽²⁾Significant reduction in pain of |
| | | intermetatarsal neuroma at week 1 and 4 |
| | | compared to placebo |
| 0.5 | 0.2 | ⁽²⁾Lowered pain scores in patients with end- |
| | | stage OA of the knee waiting for knee |
| | | replacement |
| 15 | 0.067 | ⁽³⁾Curing inguinal hernia repair improved |
| | | analgesia relative to placebo following |
| | | the first 3-4 days post surgery |

⁽¹⁾Cantilon, M. et al, Preliminary safety, tolerability and efficacy of ALGRX 4975 in Osteoarthritis (OA) of the knee, The Journal of Pain, Vol. 6, March, 2005.
⁽²⁾Diamond, E. et al, ALGRX 4975 reduces pain of intermetatarsal neuroma: preliminary results from a randomized, double-blind, placebo-controlled, phase II multicenter clinical trial, The Journal of Pain, Vol. 7, April, 2006.
⁽³⁾Aasvang, E. et al, The effect of wound instillation of a novel purified capsaicin formulation on postherniotomy pain: a double-blind, randomized, placebo-controlled study, Anesthesia and Analgesia, 2008, 107(1), p. 282-291

For several clinical trials, a high purity trans-capsaicin was supplied in vials containing 5 mL of purified capsaicin at concentrations of 0.5 mg/ml dissolved in PEG-300. The capsaicin /PEG-300 solution was stored at a temperature between 15 ° C. and 25 ° C. Within four hours prior to injection, the capsaicin concentrate was diluted to make a formulation comprising about 20% PEG-300, about 1.5 mg/ml histidine and about 5% sucrose. As noted in Table II, the capsaicin concentrations of the 3 listed examples in U.S. Pat. No. 8,420,600 are 0.002 mg/ml, 0.02 mg/ml and 0.06 mg/ml.

TABLE II
Injectable Capsaicin Dose Levels

| Capsaicin Dose | Capsaicin Dose | Total Volume of |
|---|---|---|
| Level (mg) | Concentration (mg/ml) | Injectable Dose (ml) |
| 0.01 | 0.002 | 5 |
| 0.1 | 0.02 | 5 |
| 0.3 | 0.06 | 5 |

Upon capsaicin injection in clinical trials, spontaneous burning pain and hyperalgesia was experienced immediately and persisted for up to 60 minutes. These undesirable side effects were attributed to intense activation and temporary sensitization of the peripheral nociceptors at the site of capsaicin application. This activation and sensitization occur prior to the desensitization phase.

In an attempt to control burning and stinging upon capsaicin injection, local anesthetics were injected and then withdrawn, prior to capsaicin injection.

A present limitation on the use of injectable capsaicin formulations is the likelihood of intense burning and stinging (B&S) pain for up to 1 hour and mild pain for another 1 -2 hours. The expected B&S of the capsaicin dose are believed to be from the intense nociceptor discharge occurring during the excitatory phase before nociceptor desensitization.

Work in many models has shown that capsaicin can activate sensory nerves to induce release of neuropeptides, in particular calcitonin gene-related peptide (CGRP) and the inflammatory substance P (SP)Lundberg et al, Co-existence of substance P and calcitonin gene-related peptide-like immunoreactivities in sensory nerves in relation to cardiovascular and bronchoconstrictor effects of capsaicin. Eur. J. Pharmacol., 108, 315-319, (1985). Martling et al, Calcitonin gene-related peptide and the lung: neuronal coexistence with substance P, release by capsaicin and vasodilatory effect. Regul. Pept., 20, 125-139, (1988).

It has been demonstrated that capsaicin induced edema is insensitive to treatment with the anti-inflammatory steroid, dexamethasone, which is in contrast to other forms of inflammatory edema formation. Newbold et al, An Investigation into the mechanism of capsaicin-induced edema in rabbit skin, Brit. J. of Pharma., 114, 570-577, (1995).

Ferrell et al. (Neuroscience Letters 141:259-261, 1992) demonstrated that intra-articular injection of capsaicin into the rat knee results in the significant reduction in the number of unmyelinated fibres and that this reduction was reversible.

There is an unmet need for injectable capsaicin formulations and pain treatment procedures that minimize the acute burning sensation produced following capsaicin injection. Clinical studies conducted with capsaicinoid injections have shown the ability of capsaicin to reduce pre and postsurgical pain, pain caused by osteoarthritis, tendonitis and other surgical procedures. Pain ailments are most often managed with opioids and NSAIDs and chronic use is often limited by side effects. There is an unmet need for safe and effective therapies to treat chronic pain.

### OBJECTS OF THE INVENTION

It is an object of the invention to devise formulations and methods for providing pain relief in mammals via injectable capsaicinoids that overcome the intense burning pain experienced with the administration of a capsaicinoid.

A further object of the subject invention is to provide formulations and methods of use to ameliorate or prevent the burning or stinging associated with capsaicinoid injection administration.

It is another objective of the invention to provide an optically clear solution containing aqueous and lipophilic ingredients that are totally miscible.

It is another objective of the present invention to provide formulation and methods for extended pain relief without sedation or anesthesia such as nerve, spinal or epidural blocks, for a range of therapeutic applications.

Other objects and advantages will be apparent from a review of the following specification.

### SUMMARY OF THE INVENTION

The subject invention relates to an aqueous composition for injection in a mammal comprising:
i) 0.0002%-0.1% by weight of a capsaicinoid,
ii) 99.9%-99.9998% by weight of an aqueous vehicle comprising a solubilizing agent to solubilize said capsaicinoid, and an extended release agent to slow the release of said capsaicinoid from said aqueous composition upon injection of said composition in a mammal,

wherein said aqueous vehicle reduces or eliminates the burning and stinging created by said capsaicinoid upon injection. The capsaicinoid is typically trans-capsaicin. The solubilizing agent can be polyethylene glycol, and the extended release agent is typically hyaluronic acid having an average molecular weight of about 1000 kDaltons. The extended release agent can also be collagen, elastin, and a biodegradable polymer matrix.

In an advantageous embodiment, the aqueous composition comprises:

0.005-0.05% by weight capsaicin,

5-25% by weight polyethylene glycol, and

0.2-1% by weight hyaluronic acid.

In other embodiments, the aqueous composition can include an anti-inflammatory agent, an analgesic agent, and/or a surfactant (.002-2% by wt), e.g. the nonionic surfactant polyoxy 40 hydrogenated castor oil, or PS 80. The weight ratio of capsaicinoid to polysorbate 80 can be 1 to 5.

In an advantageous embodiment the aqueous composition comprises:

0.005-0.05% by weight capsaicin,

0.025-0.15% by weight polysorbate 80 or polyoxy 40 hydrogenated castor oil,

0-25% by weight polyethylene glycol 300 or 400, and
0.2-1% by weight hyaluronic acid.

The invention also relates to a method for treating pain at a site in a mammal, e.g. human, comprising administering to said site in said mammal a therapeutically effective amount of the aqueous composition of the invention. An anesthetic is typically administered prior to the administration of said composition. In one embodiment, during the first minute after administering said capsaicinoid composition, the joint is repeatedly flexed and extended. Further, prior to, during and/or after administering said capsaicinoid composition, cooling means can be applied to the site. The capsaicinoid composition is administered in a pharmaceutically acceptable vehicle in a volume from about 0.1 ml to 25 ml.

The invention also relates to a kit for administering a capsaicinoid by intra articular injection comprising: a) at least one unit dose of an anesthetic agent, and b) at least one unit dose of a capsaicinoid solution containing a capsaicinoid and hyaluronic acid. The kit is arranged such that said anesthetic agent is administered prior to the capsaicinoid solution. The kit can include means for administration of a) and b), and can have instructions for administration.

### DETAILED DESCRIPTION OF THE INVENTION

The formulations of the invention provide a long-acting, non-opioid, treatment options for the management of moderate to severe chronic (lasting greater than 3 months) or acute pain. Effective capsaicinoid injection therapies must include treatment modalities that address the burning pain following capsaicinoid administration.

Selective and reversible defunctionalization of sensory neurons in patients with disabling chronic pain conditions by site-specific capsaicin injections is an attractive approach for long lasting (weeks to months) pain relief. However, the treatment of joints etc. with the injection of capsaicin to relieve pain is complicated by the intense burning pain capsaicin elicits upon administration.

The capsaicinoid formulations and methods disclosed herein can be utilized to treat/attenuate pain in mammals typically via injection at a discrete site to provide pain relief for an extended period of time. The formulations are administered in a pharmaceutically acceptable vehicle for infiltration. The methods and formulations further include the administration of an extended release agent, anti-inflammatory, analgesic and/or anesthetic in an amount to attenuate the burning and hyperalgesia effects of capsaicinoid administration. This can be done in conjunction with the application of cooling means, e.g.s. ice packs or gel packs, prior to, during, or subsequent to capsaicinoid injection.

Selective and reversible defunctionalization of sensory neurons by site-specific capsaicin injections provides long-lasting (weeks to months) pain relief in patients with disabling chronic pain conditions with no or minor systemic side effects. The capsaicinoid formulations of the invention alleviate or attenuate pain at the site for a prolonged period of time. With respect to pain associated with arthritic conditions such as osteoarthritis, in certain embodiments, a single unit dose capsaicinoid injection or implantation attenuates pain at the site for at least 3 months, for at least 4 months, 5 months or 6 months. With respect to joint pain, in certain embodiments, a single unit dose capsaicinoid injection or implantation attenuates pain at the site for at least one month, at least 3 months, and for 3 to 6 months, and for periods greater than 6 months, e.g. 6 to 12 months. With respect to post-surgical pain, a single unit dose capsaicinoid injection or implantation attenuates pain at the site for at least one week, and in certain embodiments for at least 1 month.

### COMPOSITIONS OF THE INVENTION

The dose of capsaicinoid is prepared for injection, implantation, infiltration or topical application by being incorporated into a pharmaceutically and physiologically acceptable aqueous vehicle for administration with diminished burning sensation upon application. The present invention is directed to the injectable administration of very small quantities of capsaicin into discrete localized areas for the treatment and lessening of pain. Significant advantages result from milligram and/or fractions of milligram quantities of capsaicin in order to produce therapeutic results through alteration of sensory nerve function (TRPV-1) function in a limited area.

The components of the compositions of the invention are discussed below.

Capsaicinoids

For a general discussion of capsaicinoids of the invention, see US patent application 2008/0262091, and commonly owned U.S. Ser. No. 13/609,100, each of which is hereby incorporated by reference in its entirety. The term “capsaicinoid” as used herein includes capsaicin, a capsaicinoid other that capsaicin, i.e. dihydrocapsaicin, nordihydrocapsaicin, homodihydrocapsaicin, homocapsaicin, and nonivamide, and a mixture of capsaicin with one or more other capsaicinoids. The amount of drug used being based on a therapeutically dose to a dose of capsaicin. Capsaicinoids are 0.0002-0.1% by wt, advantageously 0.005-0.05% by wt. of the formulations.

Alternatively, a “capsaicin analogue” such as resiniferatoxin, can be administered in place of part or all of the capsaicinoid. The amount of analogue administered being the therapeutically equivalent dose of capsaicin-see US patent application 2008/0262091, hereby incorporated by reference in its entirety. In another embodiment, a TRPV1 agonist other than a capsaicinoid, or capsaicin analogue is utilized in the formulations and methods of the invention.

Delivery Vehicles

The aqueous pharmaceutical compositions of the invention utilize pharmaceutically acceptable delivery vehicles that are single phase aqueous/water systems composed of pharmaceutically acceptable solvents such as polyethylene glycol (e.g. PEG 300 and PEG 400), ethanol and a non-ionic surfactant, e.g.s. polyoxy 40 hydrogenated castor oil (Cremophor RH40), polysorbate 80 (PS 80). An extended release agent such as hyaluronic acid controls the viscosity of the formulation and aids in formulation stability. A significant advantage of the disclosed aqueous based formulations is that water concentrations can exceed 95 wt. %.

Examples of additional components in the aqueous pharmaceutical vehicles include dextrose (sugar) and/or sodium chloride solutions to adjust osmotic pressure and tonicity, as well as buffering agents to adjust pH. The inclusion of 0.9% NaCl, 0.25% phenol, 0.25% menthol and 5% dextrose in the capsaicin/PS 80 aqueous solution did not result in a cloudy appearance or precipitate formation.

Extended Release Agents

It has been found that certain compounds which slow the release of a capsaicinoid from the formulation (causing extended release), have diminished burning and stinging effects. Advantageously, the extended release agent will release said capsaicinoid over a period of greater than 15 minutes, 30 minutes, 1 hour , or greater than 4 hours. In one embodiment, the capsaicinoid is released over a period greater than a week. Typically a higher dose will be released over a longer time period. In one embodiment of the invention, the extended release agent is not a glycosaminoglycan or a proteoglycan.

Hyaluronic Acid

The solubilization of capsaicin together with hyaluronic acid and its salts thereof, a substance that is naturally present in the human body that occurs in various tissues (skin, synovial fluids of joints and connective tissues), contributes to ameliorating the burning and stinging associated with topical and injectable capsaicin formulations.

Advantageously, 1% to 2% by weight hyaluronic acid or its salts, is used. The hyaluronic acid molecular weight used in the experimental tests described herein had an average molecular weight ranging from 800 to 1,200 kDaltons. While not wishing to be bound by theory, it is believed that the addition of the hyaluronic acid component to the capsaicin forms a polysaccharide network within the aqueous solution and causes capsaicin to be released more slowly in a controlled manner that results in a lessening of the burning and stinging pain. In one embodiment of the invention, the hyaluronic acid is in the form of a cross-linked hydrogel.

Collagen and Elastin

Collagen is the main structural protein of the various connective tissues in animals. As the main component of connective tissue, it is the most abundant protein in mammals, making up from 25% to 35% of the whole-body protein content. Collagen, in the form of elongated fibrils, is mostly found in fibrous tissues such as tendons, ligaments and skin, and is also abundant in corneas, cartilage, bones, blood vessels, the gut, and intervertebral discs.

Elastin is a protein in connective tissue that is elastic and allows many tissues in the body to resume their shape after stretching or contracting. Elastin helps skin to return to its original position when it is poked or pinched. Elastin is also an important load-bearing tissue in the bodies of vertebrates and used in places where mechanical energy is required to be stored.

Collagen and/or elastin with a capsaicinoid is advantageous in the treatment of pain including OA pain. The addition of the collagen and/or elastin component to a solubilized capsaicin formulation forms a protein network within the aqueous solution that contributes to a delayed or prolonged release of capsaicin thus contributing to minimizing the burning discomfort from either topical application or injection of capsaicin. Additionally, upon injection with a capsaicinoid these naturally occurring high molecular weight proteins function to control the rate of capsaicin release to the nerves to reduce burning, provide lubrication to the sliding bone surfaces. Addition of hyaluronic acid to capsaicinoid formulations containing either or both these naturally occurring high molecular weight proteins further optimizes tolerability and efficacy. In the compositions of the invention, the collagen or elastin must be in the form of a liquid or gel.

Bioresorbable Polymer Matrix

A bioresorbable polymer matrix, e.g. a cross-linked oxidized dextran hydrogel, can also be used in the formulations of the invention as the extended release agent. See U.S. Pat. No. 8,435,565 hereby incorporated by reference in its entirety.

Solvents

Polyethylene glycol, referred to as PEG, is used as an inactive ingredient, as a solvent, plasticizer, ointment and suppository base, and in tablets and capsules as a lubricant. PEG has low systemic toxicity with systemic absorption less than 0.5%. The term “PEG” is used, in combination with a number. Within the pharmaceutical industry, the number indicates the mean molecular weight. The low-molecular weight liquid polyethylene glycols PEG 300 and 400 are excellent solvents and co-solvents for a large number of substances that do not readily dissolve in water. They are therefore widely used as solvents and solubilizing agents for active substances and excipients in liquid and semi- solid preparations. The ability of PEGs to form complexes with active substances is responsible for their excellent solvent power. Polyethylene glycols can also be used to adjust the viscosity of liquid pharmaceutical preparations and to modify their absorption properties and to stabilize the preparations.

Polyethylene glycols with a mean molecular weight up to 400 are non-volatile liquids at room temperature. Liquid PEGs up to PEG 600 are miscible with water in any ratio. But even higher molecular weight solid PEG grades have excellent solubility in water.

The polyethylene glycols show outstanding toxicological safety regarding acute and chronic oral toxicity, embryotoxicity or skin compatibility, supported by parenteral/absorption/excretion investigations. They have been used for many years in cosmetics, foodstuffs and the pharmaceutical industries. Many of these compounds are listed on the FDA Inactive Ingredient List for use in prescription products. Formulations of the invention include up to 25% by wt solvents, typically 5-25% by wt.

An alternative to the PEG compounds is polypropylene glycol. Additionally, alcohols including ethyl alcohol, glycerol, polyethylene glycols, etc. can be added to the formulations as capsaicinoid solubilizing agents.

Surfactants

A surfactant, such as a nonionic surfactant, e.g. PS 80 and/or Cremophor® RH 40 (polyoxyl 40 hydrogenated castor oil); alternatively, Cremophor® ELP, or Solute® HS 15, can be utilized in the formulations of the invention along with a solvent (as described above) or as an alternative to a solvent. The surfactant serves as a wetting agent and emulsifier, and can lessen the initial stinging or burning discomfort associated with capsaicinoid administration.

Further, surfactant capsaicin (or other capsaicinoids) concentrates can be formed for use in the formulations and methods of the invention as described in commonly owned U.S. Pat. No. 8,637,569 hereby incorporated by reference in its entirety. These concentrates can be utilized in the formulations of the invention.

Formulations of the invention include 0.001-2.5% by wt surfactants, typically 0.1-0.5% by wt.

Analgesic and Anesthetic Agents

Analgesic ingredients are used in the formulations of the invention to ameliorate or prevent the initial acute burning or stinging pain associated with capsaicin. A variety of analgesic agents can be used in the subject invention.

Phenol and Menthol

Phenol and/or menthol can be administered at the surgical incision, open wound, or injection site to be treated. Phenol and menthol can be administered prior to administration of the capsaicinoid, or can be co-administered with the capsaicinoid. The effective 24 hour intracapsular capsular concentration for bolus injected phenol and menthol concentrations should be ˜25 μg/ml or ˜250 μg for a joint fluid injection volume of 10 ml.

Both phenol and menthol (1) rapidly penetrate the surfaces in contact with the injected formulation; and (2) serve to reduce or eliminate the acute burning and stinging pain sensation associated with the administration of the TRPV1 agonist (e.g. capsaicin). Eugenol can also be used. The subject invention includes the use of specific injectable analgesics (e.g. phenol and menthol) that have a fast “onset of action” relative to capsaicin to effectively moderate the burning sensation effect of capsaicin. Onset of action of a compound is linked to its physicochemical properties; some agents are listed in Table III.

TABLE III
Onset of Action of Selected Analgesic and Anesthetic Ingredients

| | | Oil | Aqueous | Log | MP | Onset of |
|---|---|---|---|---|---|---|
| Ingredients | MW | Soluble | Soluble | O/W | (° C.) | Action |
| Capsaicin | 305.41 | soluble | insoluble | 3.327 | 62-67 | moderate |
| Ethyl | 46.07 | soluble | miscible | −0.18 | −114 | fast |
| Alcohol | | | | | | |
| Phenol | 94.11 | soluble | soluble | 10 | 46 | fast |
| Menthol | 156.26 | soluble | slightly | 2.66 | 42 | fast |
| | | | soluble | | | |
| Lidocaine | 234.34 | soluble | insoluble | 2.359 | 68 | slow |
| Prilocaine | 220.31 | soluble | sparingly | 2.11 | 137 | slow |
| Benzocaine | 165.19 | soluble | sparingly | 1.95 | 90 | moderate |

The use of these selected analgesics with a fast onset of action effectively moderates the burning effect of capsaicin when concomitantly administered, but also provides more immediate pain relief relative to capsaicin. In one embodiment of the invention, the injected analgesic agent has a molecular weight of 160 or less. Capsaicin provides more long term/long lasting pain relief relative to these fast onset of action injectable analgesics.

Local anesthetics such as lidocaine, are common additions to intra articular injections. They are used in many basic medical procedures to produce numbness for a short period of time, including being used in doctor's offices or at hospitals to numb an area that is injured or that requires minor surgical manipulation. A local anesthetic agent can be added to the injectable vehicle to provide localized pain relief. Examples of anesthetics are lidocaine, bupivacaine, ropivacaine, dibucaine, procaine, chloroprocaine, prilocaine, mepivacaine, etidocaine, tetracaine, and xylocale. Advantageously, the anesthetic is administered prior, i.e. 2-20 minutes, to the capsaicinoid administration due to differences in the onset of action of the compounds.

Anti-Inflammatory Agents

An anti-inflammatory agent is optionally included in the formulation.

Non-Steroidal Anti-Inflammatory Drugs (NSAIDs)

Non-steroidal anti-inflammatories, such as aspirin, naproxin, indomethacin, diclofenac sodium, refecoxib, and ibuprofen, inhibit the enzyme cyclooxygenase (COX-2 inhibitor) and therefore decrease prostaglandin synthesis. Prostaglandins are inflammatory mediators that are released during allergic and inflammatory processes. In whole, the NSAIDs prevent the prostaglandins from ever being synthesized, reducing or eliminating the pain.

An NSAID anti-inflammatory agent, such as diclofenac, aspirin, ibuprofen, naproxen, benoxaprofen, flurbiprofen, fenoprofen, and others, can be added to the injectable composition to provide localized pain relief with minimal or no systemic absorption.

Corticosteroids

In one embodiment of the invention, an adjunctive agent such as a corticosteroid (glucocorticoid) is administered prior to, or concurrently with, capsaicin to attenuate an initial acute burning and stinging pain from the administered dose of capsaicin. Some of the most potent anti-inflammatories are the corticosteroids. Corticosteroids have been shown to reduce inflammation by inhibiting the production of substances that cause inflammation. The use of injectable corticosteroids is widespread in pain management. These drugs can diminish or eliminate painful foci by virtue of their anti-inflammatory properties. Corticosteroid injection can be highly effective because it delivers the medication directly to the site of inflammation. The corticosteroid injection is an effective way to alleviate inflammation including that resulting from the inflammatory substances from the TRPV-1 nerve endings due to the presence of capsaicin. While not wishing to be bound by theory, it is believed that administration of the corticosteroids works by calming nerves and reducing the inflammatory effects of certain bio- transmitters, such as substance P and bradykinin, resulting from the capsaicinoid administration.

There are several corticosteroids that can be used including dexamethasone, methylprednisolone acetate, methylprednisolone sodium succinate and mixtures thereof. Other equivalent corticosteroids known to those skilled in the art can also be used. In one embodiment, the corticosteroid solubility is increased by the formation of a corticosteroid concentrate (e.g. using PS 80 or polyoxy 40 hydrogenated castor oil) according to U.S. Pat. No. 8,637,569 hereby incorporated by reference in its entirety.

Injectable corticosteroids are available in either water-soluble or depot formulations. Water-soluble corticosteroids are typically not used for intra-articular injections because they rapidly diffuse from the injected area, exerting systemic effects. Depot formulations remain for a longer period of time at the injected site, maintaining a local effect, and are advantageous for joint injections. There are a variety of depot corticosteroids available for use including methyl prednisolone acetate, betamethasone sodium phosphate, betamethasone acetate, hydrocortisone acetate, prednisolone tebulate, triamcinolone acetonide, triamcinolone hexacetonide and mixtures thereof. Corticosteroids with lower solubility compared to compounds with greater solubility have an added benefit of maintaining effective synovial levels for a longer time and produce lower systemic levels.

When a local corticosteroid is administered with the capsaicinoid, the administration provides burning and stinging pain relief to the area to be treated. The administration of the corticosteroid with the administration of capsaicin or capsaicin-like compounds results in less pain upon capsaicin administration, and pain relief at the site for a prolonged period of time—greater than 1 month, advantageously greater than 3 months, and most advantageously, greater than 6 months.

The use in orthopedic surgery of corticosteroids with their potent anti-inflammatory activity, with capsaicinoids, eliminates or substantially reduces the acute pain from capsaicinoid administration. This allows extended pain relief in a safe and effective manner.

Aqueous Based Capsaicin Injectable Formulations—Micellar/Free

The formulations cited in Table IV rely on the PEG and ethyl alcohol to solubilize capsaicin and the analgesic agents, phenol and menthol. A nominal content of 15-40 wt. % PEG and ˜0-40 wt. % ethyl alcohol are required to maintain a single-phase aqueous solution of phenol and menthol.

TABLE IV
High PEG 300/400 Capsaicin Formulations (micellar free)

| | | CONCENTRATION | Preferred |
|---|---|---|---|
| INGREDIENT | FUNCTION | RANGE - (wt. %) | Conc. (wt. %) |
| Capsaicin | Defunctionalization of | 0.0002-0.1 | ~0.01 |
| | TRPV-1 sensory neurons | | |
| PEG 300/400 | Solubilizing agent | 15-20 | ~30 |
| Hyaluronic Acid | Viscosity enhancer, | 0.25-1.5 (can vary the | ~1.0 (1,000 |
| | stabilizing & | range of Molecular weights | kDaltons) |
| | moisturizing agent | to achieve desired | |
| | | viscosity preferably | |
| | | 800-1,500 kDaltons) | |
| Phenol, USP | Analgesic/anesthetic and | 0-1 | ~0.1 |
| | antiseptic agent | | |
| Eugenol, USP | Analgesic/anesthetic and | 0-1 | ~0.1 |
| | antiseptic agent | | |
| Menthol, USP | TRPV-8 Cooling & | 0-1 | ~0.1 |
| | analgesic agent | | |
| Sodium Chloride/ | Tonicity agents | NaCl - <0.9 | NaCl - ~0.9 |
| Sucrose | | Sucrose - ~5 | Sucrose - 5 |
| Water | Solvent (pyrogen free) | q.s. | q.s. |
| Phosphate Buffer/ | pH Control | Adjust pH from 7.0-8.0 | ~7.2 |
| Citrate Buffer | | | |
| Diclofenac Sodium | Anti-inflammatory agent | 0-1 | ~<1 |
| (a NSAID) - optional | | | |

Aqueous Based Capsaicin Injectable Formulations—Containing Capsaicin in Micelles

As noted in Examples V and Examples VI A to VI D of U.S. Pat. No. 8,637,569, compositions containing lmg/ml of capsaicin within an aqueous solution required 10 mg/ml of PS80 in aqueous solutions. It was also noted that for each 10 mg/ml increase in the PS 80 concentration that the aqueous solubility of capsaicin increases by about 1 mg/ml.

The examples of commonly owned U.S. Pat. No. 8,637,569 however, unexpectedly teach requiring one-half (½) the PS 80 concentration within an aqueous solution; i.e., 5 mg/ml of PS 80 to solubilize lmg/ml of capsaicin. The proportional 1 mg/ml concentration in the relatively aqueous insoluble capsaicin achieved with 5 mg/ml of PS 80 indicates that capsaicin is contained within micelles. (See Example 1) Therefore, to achieve a capsaicin concentration of 0.06 mg/ml, only 0.3 gm/ml of PS 80 is required.

The formulations cited in Table V below rely on the PS 80 to dissolve capsaicin via solubilizing and micellar formation and the PEG and ethyl alcohol content to solubilize the analgesic agents, phenol and menthol. A 5-20 wt. % of PEG and 0-40 wt. % ethyl alcohol are required to maintain a single-phase aqueous solution of phenol and menthol.

TABLE V
PS 80 Capsaicin Formulations

| | | CONCENTRATION | Preferred |
|---|---|---|---|
| INGREDIENT | FUNCTION | RANGE - (wt. %) | Conc. (wt. %) |
| Capsaicin | Defunctionalization of | 0.0002-0.1 | ~0.01 |
| | TRPV-1 sensory neurons | | |
| Polysorbate 80 | Capsaicin Solubilizing via | 0.001-10 | ~0.5 |
| | micelles | | |
| PEG 300/400 | Solubilizing agent | 5-20 | ~15 |
| Hyaluronic Acid | Viscosity enhancer, | 0.25-1.5 (can vary the | ~1.0 (1,000 |
| | stabilizing & | range of Molecular weights | kDaltons) |
| | moisturizing agent | to achieve desired | |
| | | viscosity preferably | |
| | | 800-1,500 kDaltons) | |
| Phenol, USP | Analgesic/anesthetic and | 0-1 | ~0.1 |
| | antiseptic agent | | |
| Eugenol, USP | Analgesic/anesthetic and | 0-1 | ~0.1 |
| | antiseptic agent | | |
| Menthol, USP | TRPV-8 Cooling & | 0-1 | ~0.1 |
| | analgesic agent | | |
| Sodium Chloride/ | Tonicity agents | NaCl - < 0.9 | NaCl - ~0.9 |
| Sucrose | | Sucrose - ~5 | Sucrose - 5 |
| Water | Solvent (pyrogen free) | q.s. | q.s. |
| Phosphate Buffer/ | pH Control | Adjust pH from 7.2-8.0 | ~7.2 |
| Citrate Buffer | | | |
| Diclofenac Sodium | Anti-inflammatory agent | 0-1 | ~<1 |
| (a NSAID) - optional | | | |

The formulations cited in Table VI below rely on Cremophor RH 40 (alternatively, Cremophor® ELP, or Solute® HS 15) to solubilize capsaicin and the analgesic agents, phenol and menthol.

TABLE VI
Cremophor ® RH 40 Capsaicin Formulations

| | | CONCENTRATION | PREFERRED |
|---|---|---|---|
| INGREDIENT | FUNCTION | RANGE - (wt. %) | CONC. (wt. %) |
| Capsaicin | Defunctionalization of | 0.0002-0.1 | ~0.01 |
| | TRPV-1 sensory neurons | | |
| Cremophor ® RH 40 or | Solubilizing Complexing | 0.001-1.0 | ~0.06 |
| Cremophor ® ELP, or | Agents via micelle | | |
| Solute ® HS 15 | formation | | |
| Hyaluronic Acid | Viscosity enhancer, | 0.25-1 (can vary the | ~0.5 (~1,000 |
| | Stabilizing and | range of Molecular weights | kDaltons) |
| | moisturizing agent | to achieve desired | |
| | | viscosity preferably | |
| | | 800-1,500 kDaltons) | |
| Phenol, USP | Analgesic/anesthetic and | 0-1 | ~0.1 |
| | antiseptic agent | | |
| Menthol, USP | Analgesic/anesthetic and | 0-1 | ~0.1 |
| | antiseptic agent | | |
| Eugenol, USP | Analgesic/anesthetic and | 0-1 | ~0.1 |
| | antiseptic agent | | |
| Sodium Chloride/ | Tonicity agents | NaCl - <0.9 | NaCl - ~0.9 |
| Sucrose | | Sucrose - ~5 | Sucrose - ~5 |
| Water | Solvent (pyrogen free) | q.s. | q.s. |
| Phosphate Buffer/ | pH Control | Adjust pH from 7.2-8.0 | ~7.2 |
| Citrate Buffer | | | |
| Diclofenac Sodium | Anti-inflammatory agent | 0-1 | ~<1 |
| (a NSAID) - optional | | | |

Methods of Making the Formulations of the Invention

Methods of making the formulations of the invention are discussed in Examples 1-6 below.

Methods of Using the Compositions of the Invention and Administration

The present invention provides for administration of an aqueous capsaicinoid formulation for treatment of pain. A single dose of the capsaicinoid formulation is administered at a discrete site, a surgical site or open wound in an amount effective to denervate the injection site, surgical site (e.g. laparoscopy) or wound (e.g. bone fracture or torn ligament). Examples of sites are joints, muscles, tendons, nerves or tumors.

The formulations of the present invention are useful in 1) relieving pain at an intra- articular site or at a body space; 2) alleviating the post surgical pain experienced by patients following discharge from a clinical care facility; 3) providing effective post- surgical analgesia such that the amount of narcotics taken by a patient is reduced thereby decreasing post-surgical rehabilitation time.

Uses and methods and of the compositions of the inventions include but are not limited to orthopedic disorders of the knee, shoulder, hip, back, spine, neck, elbows, hand, foot and other disorders which involve pain at a specific site. Examples of intra-articular administration include injection to the knee, elbow, hip, carpal, tarsal, wrist, intervertebral disk, ankle, and any other joints subject to arthritic conditions. Examples of body spaces include bursae or peritoneum.

Osteoarthritis (OA) is associated with a loss of cartilage. Cartilage is composed of specialized cells called chondrocytes that produce a large amount of extracellular matrix composed of collagen fibers, abundant ground substance rich in proteoglycan, and elastin fibers.

The compositions of the invention can be used for management and prolonged relief of nociceptive pain in mammals associated with osteoarthritis, rheumatoid arthritis, tendonitis, bursitis, pre- and post-surgical conditions, as well as for neuropathic pain occurring secondary to nerve injury. Other uses and methods of administration are described in U.S. Pat. No. 8,420,600, and commonly owned U.S. Ser. No. 13/609,100 each of which is hereby incorporated by reference in its entirety.

As used herein, “injection” means administration to a site through the skin. “Implantation” shall mean administration at a site by embedding a dose of material into the skin, muscle, tendon or joint.

The capsaicin formulations can be administered via injection or infiltration to a site. For surgery or wounds, the dose is administered to the muscle, tissue or bones surrounding the surgical or wound site. When the capsaicinoid is administered by infiltration, the capsaicinoid is administered to the surgical site or wound with an instrument known to those skilled in the art for administering via infiltration, e.g. a needle and syringe.

As used herein, “therapeutically effective amount” refers to that quantity or dose of an agent to produce a clinically desired result such as a biological response, or a reduction of a symptom of a disease or condition, e.g. reduction in or elimination of pain.

When the single dose of capsaicin is administered via injection, the injection volume of capsaicin depends on the localized site of administration. Suitable injection volumes to be delivered advantageously range from about 0.1 to about 20 ml, more advantageously from about 0.5 to about 10 ml and most advantageously from about 1.0 to about 5 ml, depending on the site to be treated.

Advantageous use of the injectable compositions of the invention for treatment of joint pain and to minimize or eliminate the burning and stinging effects of injectable capsaicin follows. Sterile technique must be used for injections in order to reduce the risk of infection. The skin is initially cleaned with Betadine or other suitable antiseptic agent.

Injection Procedure

The following procedures can be used for injection of capsaicin formulation to a joint (e.g. knee):
1. A needle is inserted into the joint and excess fluid is withdrawn.
2. An anesthetizing agent (1% Lidocaine) is injected into the affected joint and allowed to infiltrate the surrounding intra-articular surfaces for about 5 to 10 minutes. During this time period, the subject is asked to walk, flex and extend (bend) the knee to distribute the Lidocaine. Advantageously, the anesthetic agent is not removed from the joint prior to administration of the capsaicinoid formulation. A cooling means such as an ice pack or gel pack is optionally applied to the affected knee prior to, during, and/or after the Lidocaine injection.
3. Five to 10 minutes following the Lidocaine injection, the capsaicin formulation is injected at a dose of 0.2 to 0.5 mg in 5 ml solution. Ice packs or gel packs or a cooling device are optionally applied to the affected knee prior to, during and after the capsaicin formulation injection. To distribute the capsaicin in the joint, the knee is fully flexed and extended repeatedly for the first 1 minute (advantageously 30 seconds) while the subject is on the examination table. The subject is also asked to walk and flex the knee.

Flexing of Joint after Capsaicin Injection

Significantly, movement of the joint by bending (flex and extend) the joint and/or walking within the first minute after capsaicin administration serves to circulate the formulation and reduce the burning and stinging pain.

Cooling

Cooling the joint or administration site via external cooling means such as gel pack or ice pack or cooling device also reduces the painful effects from capsaicinoid administration. In one embodiment, the cooling or gel pack produces a temperature above freezing, e.g. 38-50 degrees F.

The methods of treatment of the invention utilize capsaicin (e.g. trans capsaicin) dose ranges from 0.001 mg to 1.0 mg., advantageously from 0.2 mg. to 0.4 mg. The methods utilize an aqueous vehicle in a volume from about 0.1 ml to 25 ml, advantageously 5-10 ml. The amounts of capsaicinoid and vehicle used for small joint or non-joint applications will vary as determined by a person skilled in the art.

The compositions of the invention typically include 0.0002%-0.1% by weight of a capsaicinoid, advantageously 0.01-0.05% by weight capsaicinoid.

The formulations and methods of the subject invention provide pain relief for 2 to 5 days, advantageously 7 to 21 days, or more advantageously 6 to 8 weeks, or greater than 14 weeks (i.e. greater than 1 month, advantageously greater than 3 months, and most advantageously, greater than 6 months).

Similar techniques can be used for the relief of pain relief other than joint pain.

The following examples are illustrative, but not limiting of the compositions and methods of the present invention. Other suitable modifications and adaptations of a variety of conditions and parameters normally encountered that are obvious to those skilled in the art are within the spirit and scope of the invention.

### EXAMPLE 1

### Preparation of a Concentrated Capsaicin/PS 80 Solution

U.S. Pat. No. 8,637,569 teaches 5 mg/ml of PS 80 are required to solubilize lmg/m1 of capsaicin; i.e., to achieve a capsaicin concentration of 0.06 mg/ml, only 0.3 gm/ml of PS 80 are required. Also, it was determined that the relatively aqueous insoluble capsaicin is contained within micelles since for each 1 mg/ml increase in the capsaicin solubility, 5 mg/ml of PS 80 was required. The teachings of this Patent were utilized in solubilizing capsaicin in a high purity PS 80 obtained from Croda Inc.

### STEP I—THE PREPARATION OF A CAPSACIN and PS 80 CONCENTRATE

Ingredients Include:
10 grams of Polysorbate 80 (PS 80), Super refined, Croda Inc., CAS # 9005-65-6
2 grams of Trans-Capsaicin, Aversion Technologies Inc., USP 30, 95.7% Trans-Capsaicin, Balance Cis-Capsaicin

Procedure:
1. 10 grams of PS 80 are added to a 50 ml “Pyrex” glass vial.
2. 2 grams of Trans-Capsaicin are added to the PS 80 in Step 1.
3. The mixture from Step 2 is heated to ˜125 ° C. to dissolve the Trans-Capsaicin and form the Trans-Capsaicin/PS80 concentrate.

The addition of a few capsaicin particulates to the room temperature Capsaicin/PS 80 solution did not result in capsaicin precipitates being formed thus indicating that the capsaicin solution was not supersaturated. Unexpectedly high concentrations levels were achieved; i.e., 200 mg capsaicin in 1.0 ml of PS 80 (200 mg/ml). The addition of 2 ml of this capsaicin/PS 80 concentrate to 98 grams of 70° C. water resulted in a cloudy solution which gradually became crystal clear as the solution mixture cooled to room temperature. Thus the addition of this 20% capsaicin/PS 80 concentrate to water resulted in an aqueous capsaicin solubility level of 4 mg/ml. Similarly, the addition of 10 ml of this capsaicin/PS80 concentrate to 90 grams of 70° C. water resulted in a cloudy solution which gradually became crystal clear as the solution mixture cooled to room temperature. Thus, the addition of this 2% capsaicin/PS 80 concentrate to water resulted in an aqueous capsaicin solubility level of 20 mg/ml.

It was also noted that for each 5 mg/ml increase in the PS 80 concentration that the aqueous solubility of capsaicin increases by about 1 mg/ml. While not wishing to be bound by theory, this suggests that the capsaicin is contained in PS 80 micelles.

### EXAMPLE 2

### Preparation of a 200 gram Aqueous Solution Containing the Capsaicin/PS80 Concentrate from Example 1 and Hyaluronic Acid

### STEP I—THE PREPARATION OF THE HYALURONIC ACID SOLUTION

Ingredients Include:
1 gram of Hyaluronic Acid, M. W.=1,000 kDaltons, Lotioncrafter LLC, CAS # 9067-32-7
191.8 gram of Distilled Water, Poland Springs

Procedure:
1. Add 1 gram of Hyaluronic Acid in a 400 cc Pyrex beaker.
2. Add 191.8 grams of water to the Hyaluronic Acid and thoroughly mix until a clear solution is obtained. It takes an extended time duration to completely solubilized the hyaluronic acid.

### STEP II—THE PREPARATION OF THE 0.6 mg/ml Capsaicin, 3.0 mg/ml PS80 & 5 mg/ml Hyaluronic Acid Aqueous Solution

Ingredients Include:
7.2 grams of the Capsaicin/PS 80 concentrate from Example 1.
192.8 grams of the Hyaluronic Acid from STEP I.

Procedure:
1. To the 192.8 grams of the Hyaluronic Acid Solution prepared in STEP I and contained within a 400 cc Pyrex beaker, slowly add 7.2 grams of the Capsaicin/PS 80 concentrate prepared in Example lwhile thoroughly stirring.
2. The mixture from Step 1 is now ready for subsequent packaging.

After thoroughly mixing the ingredients, the resulting mixture was crystal clear and was moderately viscous. The viscosity of the mixture can be adjusted by varying the hyaluronic acid content.

The elevated temperature solubilization of capsaicin in PS 80 is important. Several attempts to add the PS 80 and capsaicin to water followed by heating to temperatures of ˜70° C.-90° C. resulted in only a fraction of the capsaicin dissolving.

The ability to achieve increased capsaicin solubility levels within an aqueous medium as described in the foregoing text, allows for significant reductions in the liquid volume of capsaicin to be injected. Experiments with PEG-400 did not achieve the capsaicin solubility levels when compared to the PS80 (micellar solubilization) surfactant.

### EXAMPLE 3

### Preparation of 200 grams of a 0.06 mg/ml Capsaicin Injectable Solution (micelle free)

### STEP I—THE PREPARATION OF A CAPSACIN, PHENOL, MENTHOL SOLUTION

Ingredients Include:
60 grams of PEG-300, Spectrum Chemical, NF, CAS # 25322-68-3
16 grams of Ethyl Alcohol, Graves Grain Alcohol, 190 Proof
0.2 grams of Phenol, Liquefied (carbolic Acid), Spectrum Chemical, USP, CAS # 108-95-2
0.2 grams of L-Menthol, Crystal, Spectrum Chemical, USP, CAS # 2216-51-5
0.012 grams of Trans-Capsaicin, Aversion Technologies Inc., USP 30, 95.7% Trans-Capsaicin, Balance Cis-Capsaicin

Procedure:
1. Add 60 grams of PEG-300 in a 400 cc Pyrex beaker.
2. Add 16 grams of Ethyl Alcohol to the PEG-300 & thoroughly mix.
3. Add 0.2 grams of Liquefied Phenol to the mixture of Step 2.
4. Add 0.2 grams of L-Menthol Crystals to the mixture of Step 3.
5. Add 0.012 grams of Trans-Capsaicin to the mixture of Step 4
6. Heat the mixture of Step 5 to ˜40 ° C. to hasten the formation of the solution of menthol and capsaicin.
7. The solution from Step 6 is set aside and allowed to cool to room temperature.

### STEP II—THE PREPARATION OF THE HYALURONIC ACID SOLUTION

Ingredients Include:
1 gram of Hyaluronic Acid, M.W. =1,000 kDaltons, Lotioncrafter LLC, CAS # 9067-32-7
122.6 gram of Distilled Water, Poland Springs

Procedure:
1. Add 1 gram of Hyaluronic acid in a 250 cc Pyrex beaker.
2. Add 122.6 grams of water to the Hyaluronic Acid and thoroughly mix until a clear solution is obtained. It takes an extended time duration to completely solubilized the hyaluronic acid; mostly likely 30 minutes.

### STEP III—THE COMBINING OF STEP I & STEP II SOLUTIONS

Ingredients Include:
The solution mixture from STEP I
The solution mixture from STEP II.

Procedure:
1. The hyaluronic acid solution from STEP II is slowly added to the capsaicin containing solution from Step I while thoroughly stirring.
2. The mixture from Step 1 is now ready for subsequent packaging.

Table 6 contains the ingredients contained within the 0.06 mg/ml capsaicin solution.

### EXAMPLE 4

### Preparation of 200 grams of a 0.06 mg/ml Capsaicin/PS 80 Micelle Injectable Solution

### STEP I—THE PREPARATION OF A CAPSACIN and PS 80 SOLUTION

Ingredients Include: (Note: To ensure weighing accuracy, the amount of the Capsaicin/PS 80 solution prepared was 8 times that required for a 0.06 mg/ml injectable solution)
2 grams of Polysorbate 80 (PS 80), Super refined, Croda Inc., CAS # 9005-65-6
0.125 grams of Trans-Capsaicin, Aversion Technologies Inc., USP 30, 95.7% Trans-Capsaicin, Balance Cis-Capsaicin

Procedure:
1. 2 grams of PS 80 are added to a 16 ml Pyrex glass vial.
2. 0.125 grams of Trans-Capsaicin are added to the PS 80 in Step 1.
3. The mixture from Step 2 is heated to ˜55 ° C. to dissolve the Trans-Capsaicin and form the Trans-Capsaicin/PS 80 solution.

### STEP II—THE PREPARATION OF THE CAPSAICIN/PS 80, PEG-300, ETHYL ALCOHOL, PHENOL & MENTHOL SOLUTION

Ingredients Include:
20 mg of PEG PEG-300, Spectrum Chemical, NF, CAS # 25322-68-3
10 grams of Ethyl Alcohol, Graves Grain Alcohol, 190 Proof
0.27 grams of the Trans-Capsaicin/PS 80 solution from STEP I.
0.2 grams of Phenol, Liquefied (carbolic acid), Spectrum Chemical, USP, CAS #108-95-2
0.2 grams of L-Menthol, Crystal, Spectrum Chemical, USP, CAS # 2216-51-5

Procedure:
1. Add 20 grams of PEG-300 in a 400 cc Pyrex beaker.
2. Add 10 grams of Ethyl Alcohol to the PEG-300 and thoroughly mix.
3. Add 0.27 grams of the Trans-Capsaicin/PS 80 solution to the mixture from Step 2 and thoroughly stir.
4. Add 0.2 grams of Liquefied Phenol to the mixture of Step 3.
5. Add 0.2 grams of L-Menthol Crystals to the mixture of Step 4.
6. Heat the mixture from Step 5 to ˜40 ° C. to hasten the solution of menthol.
7. The solution from Step 5 is set aside and allowed to cool to room temperature

### STEP III—THE PREPARATION OF THE HYALURONIC ACID SOLUTION

Ingredients Include:
1 gram of Hyaluronic Acid, M.W. =1,000 kDaltons, Lotioncrafter LLC, CAS #9067-32-7
169.5 grams of Distilled Water, Poland Springs

Procedure:
1. Add 1 gram of Hyaluronic acid in a 400 cc Pyrex beaker.
2. Add 169.5 grams of water to the Hyaluronic Acid and thoroughly mix until a clear solution is obtained. It takes an extended time duration to completely solubilized the hyaluronic acid.

### STEP III—THE COMBINING OF STEP I & STEP II SOLUTIONS

Ingredients Include:
The solution mixture from STEP I
The solution mixture from STEP II.

Procedure:
1. The hyaluronic acid solution from STEP II is slowly added to the capsaicin containing solution from STEP II while thoroughly stirring.
2. The mixture from Step 1 is now ready for subsequent packaging.

Table 6 contains the ingredients contained within the 0.06 mg/ml capsaicin solution.

### EXAMPLE 5

Preparation of 200 grams of a 4 mg/ml Capsaicin, 1 mg/ml Menthol and 1 mg/ml Phenol Transparent Aqueous Concentrate

The following steps were followed in the preparation of a 4 mg/ml Capsaicin, 1 mg/ml Menthol and 1 mg/ml Phenol concentrate.

Ingredients Include:
0.40 grams of the Trans-Capsaicin, Aversion Technologies Inc., USP 30, 95.7% Trans-Capsaicin, Balance Cis-Capsaicin
0.10 grams of Phenol, Liquefied (carbolic acid), Spectrum Chemical, USP, CAS # 108-95-2
0.10 grams of L-Menthol, Crystal, Spectrum Chemical, USP, CAS # 2216-51
2 grams Cremophor® RH 40, CAS Number 61788-85-0 obtained from Sigma Aldrich, St. Louis, Mo.

Procedure:
1. Add 0.4 grams of capsaicin powder, 0.1 grams of menthol crystals and 0.1 grams of liquefied phenol to a 300 cc Pyrex beaker.
2. Add 2.0 grams of Cremophor° RH 40 to the mixture from Step 1.
3. The mixture from Step 2 is heated to about 125° C.
4. Water is slowly added to the mixture from Step 3 while thoroughly stirring to provide an aqueous mixture weighing 200 grams.

An optically clear aqueous solution containing 4 mg/ml Capsaicin, 1 mg/ml Menthol, 1 mg/ml Phenol and 20 mg/ml Cremophor° RH 40 resulted. A combined weight ratio of the capsaicin, menthol and phenol mixture to the Cremophor° 40 surfactant was 1.0/3.33.

### EXAMPLE 6

Preparation of the HA/PEG/Capsaicin Injection Solution

The capsaicin used in the injectable formulation is a high purity synthetic Trans-Capsaicin product intended for drug use. Formosa Laboratories is the certified manufacturer (DMF #16769). Johnson Compounding of Waltham, Mass. prepared the capsaicin injection formulation as a sterile liquid and Bioventus LLC of Durham, N.C. supplied the sterile hyaluronic acid (HA), Supartz, formulation used for preparing the sterile capsaicin-HA injection solution. The capsaicin solution and Supartz were manufactured and released in compliance with Good Manufacturing Practices (GMPs). The capsaicin solution is manufactured by dissolving capsaicin in 100% polyethylene glycol 300 (PEG). The resulting solution was sterile filtered and aseptically filled into 5 ml Type I glass vials that that were subsequently sealed. The components and compositions of the capsaicin solution are listed in Table VII.

TABLE VII
Components of the Capsaicin Solution

| Ingredients | Concentration | Function |
|---|---|---|
| 99⁺ wt % trans-capsaicin¹ | 0.04 wt % | API |
| | (0.4 mg/ml) | |
| PEG-300 Ph Eur² | q.s. ad | Solvent |

¹The cGMP 99⁺ wt % trans-capsaicin was procured from Formosa Laboratories, Taiwan
²Sigma-Aldrich, St. Louis, MO (Catalog #81162)

The capsaicin solution was then mixed with Supartz. Supartz is a sterile, viscoelastic non- pyrogenic solution of purified, high molecular weight (620,000-1,170,000 Daltons) sodium hyaluronate (hyaluronan) having a pH of 6.8-7.8. Each one ml of Supartz contains 10 mg of sodium hyaluronate (hyaluronan) dissolved in a physiological saline (1.0% solution). The sodium hyaluronate (hyaluronan) is extracted from chicken combs. Sodium hyaluronate (hyaluronan) is a polysaccharide containing repeating disaccharide units of glucuronic acid and N-acetylglucosamine. The components and composition of Supartz are shown in Table VIII.

TABLE VIII
Components of Supartz

| Ingredients | Concentration (wt %) | Function |
|---|---|---|
| Sodium Hyaluronate | 1.0 | Solvent |
| (hyaluronan) | | |
| Sodium Chloride | 0.85 | Isotonic Agent |
| Dibasic Sodium Phosphate | 0.05 | Buffering Agent |
| Dodecahydrate | | |
| Sodium Dihydrogen | 0.002 | Buffering Agent |
| Phosphate Dihydrate | | |
| Water | q.s. ad | Solvent |

Capsaicin concentration of the aqueous capsaicin-HA sterile injection solution ranged from 0.2-0.5 mg of capsaicin in 5 ml sterile injection solution and was prepared by mixing the capsaicin solution with the hyaluronic acid formulation, Supartz, just prior to injection into the intra-articular knee joint.

### EXAMPLE 8

Injection of the HA/PEG/Capsaicin Solution into the Knee

A 72 year old male with painful osteoarthritis of the knees received an injection of an 8 ml solution containing 1% Lidocaine into the intra-articular cavity of his left knee. The Lidocaine solution was distributed throughout the joint cavity by walking and bending of the knee for about 5 minutes prior to intra-articular injection of 5 ml of the aqueous capsaicin-HA sterile solution. A capsaicin dose level of 0.2 mg capsaicin was achieved by mixing 0.5 ml of the capsaicin solution with 4.5 ml of the HA containing Supartz vehicle. The knee had been pre-cooled for about 3 minutes prior to the injection of the aqueous capsaicin-HA sterile solution and cooling of the knee was continued for the remainder of the treatment procedure. Immediately after the capsaicin injection, the knee was bent (flexed and extended) vigorously for about 1 minute to distribute the aqueous capsaicin-HA sterile solution within the joint cavity. The pain level briefly rose to a moderate level quickly subsided upon resting and bending the knee. Seven minutes after injection of the aqueous capsaicin-HA sterile solution, the procedures were completed and the subject experienced complete relief of his osteoarthritic pain symptoms. Table IX summaries of procedural pain levels as a function of elapsed time.

TABLE IX
INJECTED KNEE # 1
0.5 ml Capsaicin solution and 4.5 ml Supartz
(0.2 mg Capsaicin dose level)

| ELAPSED | | PAIN |
|---|---|---|
| TIME | | SCORE |
| (Minutes) | PROCEDURE | (0-10) |
| 0 | Cold pack applied to knee & | 0 |
| | Inject 8 cc 1% Lidocaine, Walk | |
| | around, flex & extend knee | |
| 7 | Inject 5 cc Cap/Supartz mixture, | 0 |
| | flex & extend knee vigorously | |
| 8 | | 2 |
| 9 | Vigorously flex & extend knee | 3 |
| | forabout 1 minute | |
| 10 | Flex & extend knee | 2 |
| 11 | | 3 |
| 12 | Flex & extend knee | 2 |
| 13 | | 1.5 |
| 14 | Walking, no pain | 0 |

Note:
Elapsed time from Capsaicin Inject to complete join pain relief = 7 minutes

### EXAMPLE 8

Injection of the HA/PEG/Capsaicin Solution into the Knee

A 74 year old male with painful osteoarthritis of the knees had 14 cc of fluid removed from his left knee joint prior to injection of an 10 ml solution containing 1% Lidocaine into the intra-articular cavity of his left knee. The Lidocaine solution was distributed throughout the joint cavity by walking and bending of the knee for about 7 minutes prior to intra-articular injection of 5 ml of the aqueous capsaicin-HA sterile solution. A capsaicin dose level of 0.3 mg capsaicin was achieved by mixing 0.75 ml of the capsaicin solution with 4.25 ml of the HA containing Supartz vehicle. The knee had been pre-cooled for about 2 minutes prior to the injection of the capsaicin-HA sterile solution and cooling of the knee was continued for the remainder of the treatment procedure. Immediately after the capsaicin injection, the knee was bent vigorously for about 1 minute to distribute the aqueous capsaicin-HA sterile solution within the joint cavity. The pain level briefly rose to a relatively high level and quickly subsided upon the application of additional icing and bending of the knee. Fourteen minutes after injection of the aqueous capsaicin-HA solution, the procedures were completed and the subject experienced complete relief of his osteoarthritic pain symptoms. Table X summarizes of procedural pain levels as a function of elapsed time.

TABLE X
INJECTED KNEE # 2
0.75 ml Capsaicin solutin & 4.25 ml Supartz
0.4 mg Capsaicin dose level

| ELAPSED | | PAIN |
|---|---|---|
| TIME | | SCORE |
| (Minutes) | PROCEDURE | (0-10) |
| 0 | Remove 14 cc fluid from the joint | 0 |
| | & Inject 10 cc of 1% Lidocaine, | |
| | Walk around | |
| 7 | Inject 5 cc Cap/Supartz mixture, | 6 |
| | flex & extend knee vigorously | |
| 8 | — | 8 |
| 10 | — | 5.5 |
| 11 | — | 4.5 |
| 12 | Additional ice applied | 3 |
| 13 | — | 3.5 |
| 14 | Flex & extend knee | 3 |
| 15 | Flex & extend knee several | 3 |
| | times | |
| 15 | — | 3 |
| 17 | — | 2.5 |
| 18 | — | 2 |
| 19 | — | 2 |
| 20 | — | 2 |
| 21 | Walking | 0 |

Note:
Elapsed time from Capsaicin Inject to complete join pain relief = 14 minutes

### EXAMPLE 9

Injection of the HA/PEG/Capsaicin Solution into the Knee

The same 74 year old male as in Example 8 with painful osteoarthritis of the knees had 5 cc of fluid removed from his right knee joint prior to injection of an 10 ml solution containing 1% Lidocaine into the intra-articular cavity of his left knee. The Lidocaine solution was distributed throughout the joint cavity by walking and bending of the knee for about 8 minutes prior to intra-articular injection of the 5 ml aqueous capsaicin-HA sterile solution. A capsaicin dose level of 0.4 mg capsaicin was achieved by mixing 1 ml of the capsaicin solution with 4 ml of the HA containing Supartz vehicle. The knee had been pre-cooled for about 2 minutes prior to the injection of the capsaicin-HA sterile solution and cooling of the knee was continued for the remainder of the treatment procedure. Immediately after the capsaicin injection, the knee was bent vigorously for about 1 minute to distribute the aqueous capsaicin-HA sterile solution within the joint cavity. The pain level briefly rose to a relatively moderate level quickly subsided upon the and bending and resting of the knee. Eleven minutes after injection of the aqueous capsaicin-HA solution, the procedures were completed and the subject experienced complete relief of his osteoarthritic pain symptoms. Table XI summarizes of procedural pain levels as a function of elapsed time.

TABLE XI
INJECTED KNEE # 3
1 ml Capsaicin solution & 4 ml Supartz
0.4 mg Capsaicin dose level el

| ELAPSED | | PAIN |
|---|---|---|
| TIME | | SCORE |
| (Minutes) | PROCEDURE | (0-10) |
| 0 | Remove 5 cc fluid from the joint | 0 |
| | & Inject 10 cc of 1% Lidocaine, | |
| | Walk around | |
| 8 | Inject 5 cc Cap/Supartz mixture, | 4 |
| | flex & extend knee vigorously | |
| 9 | — | 4.5 |
| 10 | — | 5.5 |
| 11 | — | 5 |
| 12 | — | 3.5 |
| 13 | — | 3.5 |
| 14 | — | 3 |
| 15 | — | 2.5 |
| 16 | Flex & extend knee several | 2.5 |
| | times | |
| 17 | — | 2 |
| 18 | Walking | 2 |
| 19 | — | 0 |

Note:
Elapsed time from Capsaicin Inject to complete join pain relief = 11 minutes

All documents and references cited above are hereby incorporated by reference in their entirety in this application.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. An aqueous pharmaceutical solution in unit dose form for injection in a mammal comprising:
0.001 to 1 mg capsaicin,
an aqueous vehicle comprising a solubilizing agent to solubilize said capsaicin, and hyaluronic acid,
wherein said aqueous vehicle reduces or eliminates the burning and stinging created by said capsaicin upon injection, and
wherein said hyaluronic acid is 0.25-1.5% by weight of said aqueous pharmaceutical solution.

2. The aqueous pharmaceutical solution as in claim 1, wherein said solubilizing agent is polyethylene glycol.

3. The aqueous pharmaceutical solution as in claim 1, wherein said capsaicin is trans-capsaicin.

4. The aqueous pharmaceutical solution as in claim 1, wherein said capsaicin is in the form of a concentrate with a nonionic surfactant.

5. The aqueous pharmaceutical solution as in claim 1, wherein said solubilizing agent is selected from the group consisting of polyethylene glycol-300, polyethylene glycol-400, and polypropylene glycol.

6. The aqueous pharmaceutical solution as in claim 1, wherein solubilizing agent is polyethylene glycol-300.

7. An aqueous solution as in 1 comprising 0.001-0.5 mg capsaicin.

8. The aqueous pharmaceutical solution as in claim 6, wherein said hyaluronic acid has an average molecular weight of 1000 kDaltons.

9. The aqueous pharmaceutical solution as in claim 6, wherein said hyaluronic acid is in the form of a hydrogel.

10. The aqueous pharmaceutical solution as in claim 1, wherein said hyaluronic acid is 800 to 1,200 kDaltons.

11. The aqueous pharmaceutical solution as in claim 1, wherein said solution is
5-25% by weight polyethylene glycol, and
0.25-1% by weight hyaluronic acid.

12. The aqueous pharmaceutical solution as in claim 1 further comprising a nonionic surfactant.

13. The aqueous pharmaceutical solution as in claim 12, wherein the nonionic surfactant is polyoxy 40 hydrogenated castor oil.

14. The aqueous pharmaceutical solution as in claim 12, wherein the nonionic surfactant is polysorbate 80.

15. The aqueous pharmaceutical solution as in claim 14, wherein the weight ratio of capsaicin to polysorbate 80 is 1 to 5.

16. The aqueous pharmaceutical solution as in claim 1 further comprising an anti-inflammatory agent.

17. The aqueous pharmaceutical solution as in claim 16, wherein said anti-inflammatory agent is a corticosteroid.

18. The aqueous pharmaceutical solution as in claim 1, further comprising an analgesic agent.

19. The aqueous pharmaceutical solution as in claim 18, wherein said analgesic agent is one or more selected from the group consisting of phenol, menthol and eugenol.

20. The aqueous pharmaceutical solution as in claim 1 further comprising an anesthetic agent.

21. The aqueous pharmaceutical solution as in claim 1, comprising:
0.005-0.05% by weight capsaicin,
0.025-0.15% by weight polysorbate 80 or polyoxy 40 hydrogenated castor oil,
0-25% by weight polyethylene glycol 300 or 400, and
0.25-1% by weight hyaluronic acid.

22. A method for treating pain at a site in a mammal comprising:
administering to said site in said mammal an aqueous pharmaceutical solution comprising 0.001-1 mg capsaicin, a solubilizing agent to solubilize said capsaicin, and hyaluronic acid.

23. A method as in claim 22 wherein said mammal is a human.

24. A method as in claim 22 wherein an anesthetic is administered prior to the administration of said aqueous pharmaceutical solution.

25. A method as in claim 24, wherein the anesthetic agent is administered in a pharmaceutically acceptable vehicle in a volume from about 0.1 ml to 25 ml.

26. A method as in claim 22 wherein during the first minute after administering said aqueous pharmaceutical solution, the joint is repeatedly flexed and extended.

27. A method as in claim 22 wherein prior to, during and/or after administering said aqueous pharmaceutical solution, cooling means is applied to said site.

28. A method as in claim 22 wherein said aqueous pharmaceutical solution is administered by injection.

29. A method as in claim 22 wherein said aqueous pharmaceutical solution is administered by infiltration.

30. A method as in claim 22 wherein the pain is from a wound or surgical site.

31. A method as in claim 22, wherein the capsaicin is trans-capsaicin dose level ranges from 0.001 mg to 0.5 mg.

32. A method as in claim 22, wherein said aqueous pharmaceutical solution is administered in a volume from about 0.1 ml to 25 ml.

33. A method as in claim 22 wherein said aqueous pharmaceutical solution comprises a capsaicin, hyaluronic acid, and polyethylene glycol.

34. A kit for administering a capsaicinoid by intra articular injection comprising:
a) at least one unit dose of an anesthetic agent
b) at least one unit dose of a capsaicinoid solution containing a capsaicinoid and hyaluronic acid,
wherein said kit is arranged such that said anesthetic agent is administered prior to said capsaicinoid solution.

35. A kit as in claim 34 further comprising a means for administration of a) and b).

36. A kit as in claim 34 further comprising instructions for administration.

37. A kit as in claim 34 wherein the capsaicinoid, hyaluronic acid and anesthetic are formulated together.

38. A kit as in claim 34 wherein said unit dose of a capsaicinoid is 0.001-1 mg capsaicin.

39. A kit as in claim 34 wherein said anesthetic is lidocaine.

40. A method as in claim 24 wherein said anesthetic is lidocaine.

41. A method as in claim 22, wherein said aqueous pharmaceutical solution is administered in a volume from about 0.5 ml to 10 ml.

42. A method as in claim 22 wherein said site is a joint.

43. A method as in claim 22 wherein said administration to said site is by intra articular injection to the knee.

44. An aqueous solution as in claim 1 comprising 0.2-4 mg capsaicin.

45. An aqueous solution as in claim 1 comprising 0.2-0.5 mg capsaicin in 5 ml aqueous vehicle.

46. An aqueous solution as in claim 1 comprising 0.2 mg capsaicin in 5 ml aqueous vehicle.

47. An aqueous solution as in claim 1 comprising 0.3 mg capsaicin in 5 ml aqueous vehicle.
